(19) United States
(12) Patent Application Publication
(10) Pub. No.: US 2007/0184039 A1
(43) Pub. Date: Aug. 9, 2007

(54) Process for the selective withdrawal of bacterial strains adhering to intestinal mucosa through endoscopy

(76) Inventors: Giovanni Mogna, Novara (IT); Mario Del Piano, Novara (IT); Gian Paolo Strozzi, Novara (IT); Lorenzo Morelli, Piacenza (IT)

Correspondence Address: PEARNE & GORDON LLP, 1801 EAST 9TH STREET, SUITE 1200, CLEVELAND, OH, 44114-3108, US

(21) Appl. No.: 10/566,525

(22) PCT Filed: Jul. 30, 2004
(86) PCT No.: PCT/IB04/02446

§ 371 Date: Jun. 19, 2006

(30) Foreign Application Priority Data
Aug. 1, 2003 (IT) MI2003A001594

(51) Int. Cl. A61K 35/74; C12N 1/20

(52) U.S. Cl. 424/93.45; 435/252.9

(57) ABSTRACT

The present invention relates to a novel process for collecting and isolating bacterial strains adhered to the intestinal mucosa by the endoscopic route by means of an innovative technique called “brushing”.

### SUMMARY OF THE INVENTION

The present invention relates to a novel method for collecting and isolating bacterial strains adhered to the intestinal mucosa by the endoscopic route, particularly a collecting method employing “brushes” for an innovative use, herein indicated as “brushing”.

### FIELD AND BACKGROUND OF THE INVENTION

The human gastrointestinal tract (GI) hosts several communities of bacteria, mainly strictly anaerobic, which besides performing important metabolic activities, such as the synthesis of vitamins and essential amino acids, also carry out a number of other useful functions for the host.

Up to now, both the complicated interactions existing between the various bacterial populations and the multiple influences exerted by the intestinal microbiota on humans are for the most part still obscure. However, worldwide studies and researches have disclosed that the bacterial microflora is capable of protecting against pathogenic germs, stimulating the immune system and generally inducing a number of beneficial effects on health.

It is believed that this commensal microflora, the cell amount being here about 20 times higher than in the whole human organism, may consist of a wide variety of different species (400-500), but 99% is made up by 30-40 major species.

At present time, the acquired knowledge essentially concerns the microflora present in the faeces, i.e. almost exclusively representative of the distal tract of the large intestine, which is a drawback of the techniques employed so far.

In recent years, some researches have been carried out on the bacterial communities present in samples collected “in vivo”, by biopsy, in various parts of the digestive system, mainly at the ascending, transverse and descending colon. According to the little information available, the intestinal microflora is distributed in the various tracts in a manner which is very diversified both in terms of quality and quantity.

Furthermore, it has been established that, in the same subject, the microflora adhered to the mucosa greatly differs from that in the lumen, thus emphasizing that the background art as yet available concerning the intestinal microbiota, which is almost exclusively based on the research on the bacterial populations present in the faeces, is absolutely partial and incomplete.

Actually, the existing difficulties in research can be essentially traced to two factors: the microbial complexity of the bacterial population and the complexity of carrying out “in vivo” collections from the various tracts of the small and large intestine.

The problems concerning the determination of the quality/quantity composition of the intestinal microflora being collected have been mostly solved in the last decade. In fact, if conventional microbiology techniques were previously employed which only partially could evidence, by means of selective and differential culture media, the biodiversity existing within the intestinal ecosystem, now due to the use of molecular biology techniques (PCR, ARDRA, RAPD, Ribotyping, REA, PFGE, DNA probes, etc.) all the bacteria present within a complex ecosystem can be detected, including the minority ones.

Those techniques suitable to determine the nucleotide sequence of the ribosomal RNA of 16S and 23S regions, which by consisting of hypervariable nucleotide sequence genes, have profiles characteristic of each microbial species have been particularly useful.

In very recent times the use of PCR (Polimerase Chain Reaction) combined with DGGE (Denaturing Gradient Gel Electophoresis) as well as TGGE (Temperature Gradient Gel Electophoresis), after having extracted the total DNA and subsequently amplified the rRNA relative to gene 16S, has enabled to determine the microbial composition of mixed bacterial populations and obtain a genetic fingerprinting of the whole community in a quick and cost-effective manner.

Therefore, by this analytic strategy, any modification occurring within a bacterial population variously divided into genus, species and biotypes can also be monitored and evaluated.

Conversely, poor progress has been made on the “in vivo” tissue sampling techniques.

This is a serious drawback in deepening the knowledge of the microflora adhered to the walls, which is the most important component of the entire microflora just because it is directly in contact with the intestinal epithelium and thus most involved in the preservation of the integrity of the wall cells, in the adjustment of the absorption of nutrients and water, and in the modulation of the immune system.

Therefore, there exist a number of gaps to be filled in the composition and roles of the various bacterial populations and the interaction existing between the lumen microflora and the microflora adhered to the walls, and between the adhered microflora and the epithelial cells.

Filling this information gap has become more and more important, since a growing consumer demand for commercial preparations containing living cells of microrganisms called probiotic has been witnessed in recent years.

Almost the entirety of the strains so far used in these products, comprising patent medicines, dietary preparations and food supplemented with probiotics, belongs to the Lactobacillus and Bifidobacterium genus which are almost solely isolated from faecal samples and then, as such, they are only representative of the colon lumen and sigma microflora.

It should be understood that while inducing beneficial effects on consumers—which are in many cases also supported with clinical evidence (improvement of the intestinal motility, reduction of symptoms of lactose intolerance, inhibition of noxious bacteria causing dysentery or intestinal infections, etc.), the probiotic preparations formulated with these strains cannot perform the particular functions of the entire intestinal microflora, both due to the fact that they lack a similar composition complexity and because of the absence of mucosa-specific strains.

In actual fact, these limitations have been emphasized by all the tests carried out so far, where it has been proved that 3-12 days after the intake of any probiotic strain has been stopped, the latter is no longer traceable in faeces, which means that the colonisation had only been a temporary one and had exclusively affected the intestinal lumen but not the wall.

In order to prevent and/or solve many anomalous and pathologic conditions of the gastrointestinal tract such as the functional disorders or FBD (Functional Bowel Disorders), the chronic inflammatory bowel diseases or IBD (Inflammatory Bowel Diseases), Crohn's disease, ulcerative colitis and many other serious and debilitating pathologies affecting the bowel, a more thorough study of the communities of the various tracts and mainly the microflora adhered to the mucosa is essential, as well as having all different strains of bacteria adhered to the various tracts of the bowel at one's disposal.

To manage the knowledge of the quality-quantity distribution of these strains is very important from a theoretical point of view, but is also particularly useful for devising targeted therapies.

In fact, the bacteria adhered in the various intestine districts, due to their inherent peculiarity of adhering to the mucosa, are capable, also when administered orally, to colonize the intestinal wall by positively interacting and integrating with the autochthonous species.

Besides a prompt “barrier” effect that, by reducing the intestinal permeability, prevents the infiltration of the pathogenous germs, a much more durable colonization is achieved which extends the probiotic effects beneficial for the host over time.

### DESCRIPTION OF THE INVENTION

To the above purposes, the inventors have attempted to find novel collection techniques allowing to study the microbial complexity of the adhered microbial populations and hence recover the probiotic strains characterizing the various tracts in order to prepare really effective probiotic cultures for the treatment of disorders and pathologies derived from the alteration or impoverishment of the microflora on the walls.

Thus, according to one aspect thereof, the invention relates to a novel technique of collecting mucosa samples by the multiple brushing of different segments of the intestinal wall aiming at collecting bacterial strains adhered thereto.

More particularly, the object of the invention is a process for collecting bacteria adhered to a subject's intestinal wall comprising carrying out said collection by a colonoscopy by employing suitable equipment provided with brushes adapted to the collection of bacterial strains.

According to an advantageous aspect of the present invention, the collection is carried out in various segments of the bowel by means of individual brushes.

According to another preferred aspect, the collection is carried out at least on one of the walls of the distal ileum, right, transverse, left colon and sigma, advantageously on all of the five walls mentioned above, by changing 5 brushes per each subject, advantageously washing the biopsy channel with saline before introducing each brush.

To carry out the colonoscopy used in the invention, materials and equipment are employed which are conventionally used in these methods, by first applying the brushes to collect the adhered bacteria.

Said brushes can be similar to those normally used in cytology or in the pathogen microbiological research, such as those available from Boston Scientific, Wilson Cook and Innovamedica.

After the strains have been collected, they are cultured and analysed by the techniques known by those skilled in the art. By way of example, the samples can be analysed by PCR-DGGE.

Thereby, according to another aspect thereof, the subject matter of the invention is a method for isolating bacterial strains adhered to a subject's intestinal wall comprising carrying out said collection by means of a colonoscopy employing suitable equipment provided with brushes adapted to the collection of bacterial strains, culture and identify these strains being collected.

The strains collected and identified can be then cultured to prepare patent medicines, dietary preparations or food containing them.

According to another aspect thereof, the invention also relates to the use of bacterial strains collected according to the method of the invention for preparing medicaments intended to treat and/or prevent the alterations of the intestinal microflora.

In order to be administered as medicaments, the strains collected according to the invention are preferably formulated in pharmaceutical compositions according to known techniques.

According to another aspect thereof, the invention also relates to pharmaceutical compositions comprising bacterial strains collected by the method of the invention which are intended to treat and/or prevent the alterations of the intestinal microflora.

The compositions of the invention comprise the strains preferably in a freeze-dried form and are advantageously formulated in the form of dose units, such as sachets, tablets, capsules, vials, etc.

Contrarily to what has been stated in the literature, the analysis carried out by the method of the invention has surprisingly shown the presence of some species of bifidobacteria also at the mucosa of distal small intestine.

More particularly, within the Bifidobacterium genus, the Bifidobacterium breve and Bifidobacterium longum are frequently found as species composing the microflora of the ileum wall, at least within the typology of Mediterranean population being studied.

A further and as much important evidence that emerged from the study of the adhered population, collected by the novel technique, concerns the fact that the wall colonization can be accomplished, within the same microbial species, by the same biotype. It has been possible to demonstrate this phenomenon using molecular biology techniques applied to samples collected by “brushing” from the mucosa of various tracts (ileum, right, transverse and left colon) in the same subject.

Examples thereof are a strain of Lactobacillus fermentum traced at the ileum, transverse and left colon in the subject A and a strain of Bifidobacterium breve found adhered to the mucosa of ileum, transverse and right colon of subject B.

Accordingly, due to the new collection technique a very important phenomenon to understand the relations existing within the intestinal microbial ecosystem has been disclosed, i.e. the possibility has emerged that the same biotype of lactobacillus or bifidobacterium may colonize different niches of the gastroenteric system in the same host, habitat characterized by environmental conditions that may be very different.

Based on information already acquired and still to be acquired by the practice of the inventive method, it is possible for example to conceive the formulation of specific probiotic products based on these bifidobacteria and lactobacillus, capable of adhering to the epithelial cells both in the small intestine, such as to fight Crohn's disease, which is known to mainly affect the small intestine, and in the colon to treat other forms of intestinal disorders and pathologies.

Alternatively, the strains to be obtained by the method of the invention can be used in the food industry for the preparation of probiotic foods; to the purpose, the strains can be included in various food preparations, such as dairy products, yogurt, creams, stuffing, juices, drinks, etc.

The probiotic foods comprising the strains obtained according to the method of the invention are a further subject matter of the invention.

Some techniques to be used, as well as the protocol to be used for verifying the feasibility of the method of the invention are set forth in the experimental part below. The examples given should be regarded as merely illustrative and non-limiting.

### Experimental Section

### EXAMPLE

Step 1

Method of Collection and Culture of Adhered Strains

Screening colonoscopy has been carried out on 10 healthy subjects aged 36-65 years, 4 males and 6 females, identified by letters A to L. No. 4 sample collections have been performed on each subject: at the distal ileum, right, transverse and left colon, by changing 4 brushes per each patient and washing with saline the biopsy channel before inserting each brush. The tip of each brush has been cut and placed in a sterile Petri capsule, immediately shifted to anaerobiosis conditions and to a temperature of +4° C. The different collections have been sent to the Microbiology laboratory within 3 hours.

Step 2

Strain Identification

The samples have been analysed by PCR-DGGE, according to the methods described in the literature (bibliography):

1. Temmerman R, Scheirlinck I, Huys G, Swings J. “Culture-indipendent analysis if probiotic products by denaturin gradient gel electrophoresis.” Appl Environ Microbiol 2003 January; 69(1):220-6

2. Burton J P, Cadieux P A, Reid G “Improved understanding of the bacterial vaginal microbiota of women before and after probiotic instillation” Appl Environ Microbiol 2003 January; 69 (1): 97-101

3. Burton J P, Reid G “Evaluation of the bacterial vaginal flora of 20 postmenopausal women by direct (Nugent score) and molecular (polymerase chain reaction and denaturing gradient gel electrophoresis) techniques” J Infect Dis. 2002 December. 15; 186 (12):1770-80 Epub 2002 November. 22.

4. McCartney A L. “Application of molecular biological methods for studying probiotics and the gut flora” Br J Nutr. 2002 September; 88 Suppl 1:S29-37 Review.

5. Zoetendal E G, von Wright A, Vilpponen-Salmela T, Ben-Amor K, Akkermans A D, de Vos W M. “Mucosa-associated bacteria in the human gastrointestinal tract are uniformly distributed along the colon and differ from the community recovered from feces.” Appl Environ Microbiol. 2002 July; 68(7):3401-7

6. HeiligHG, Zoetendal E G, Vaughan E E, Marteau P, Akkermans A D, de Vos W M. “Molecular diversity of Lactobacillus spp. And other lactic acid bacteria in the human intestine as determined by specific amplification of 16s ribosomal DNA.” Appl Environ Microbiol. 2002 January; 68(1):114-23

7. Walter J, Hertel C, Tannock G W, Lis C M, Munro K, Hammes W P.“Detection of Lactobacillus, Pediococcus, Leuconostoc, and Weissella species in human feces by using group- specific PCR primers and denaturing gradient gel electrophoresis.” Appl Environ Microbiol. 2001 June; 67(6):2578-85.

The analysis results exhibited:

in No. 5 subjects bifidobacteria adhered to the mucosa of the distal small intestine have been found, and particularly

in No. 3 cases, the colonization was operated by the Bifidobacterium breve, whereas in No. 2 cases the species found was the Bifidobacterium longum

in No. 7 subjects the same biotype of lactobacillus or bifidobacterium has been found in different bowel districts. Particularly, referring to the Lactobacillus fermentum species biotype 1 has been found in subject A at the distal ileum, transverse and right colon; biotype 2 has been found in subject C at the right, transverse colon and left colon; biotype 3 in subject F at the distal ileum, right and transverse colon; finally, biotype 4 has been found in subject G at the distal ileum, right, transverse colon and left colon. Referring to the Bifidobacterium breve species: biotype 1 has been found in subject B at the distal ileum, right and transverse colon; biotype 2 has been found in subject D at the right, transverse colon and left colon; biotype 3 in subject E at the right and transverse colon.

1. A process for collecting bacteria adhered to the intestinal wall of a subject, characterized in that said collection is carried out by means of a colonoscopy through brushes adapted to cell collection.

2. The process according to claim 1, characterized in that said brushes are connected to conventional colonoscopy equipment.

3. The process according to claim 1, characterized in that said collection is carried out in several intestine segments by means of individual brushes.

4. The process according to claim 3, characterized in that said collection is carried out at least on one of the walls of the distal ileum, right, transverse colon and left colon.

5. The process according to claim 4, characterized in that said collection is carried out on the walls of the distal ileum, right, transverse colon and left colon.

6. The process according to claim 1, characterized in that the strains collected are cultured and analysed.

7. A process for isolating bacterial strains, comprising collecting said strains according to claim 1.

8. Use of probiotic bacteria strains obtained by the process of claim 1, for preparing medicaments to treat and/or prevent the alterations of the intestinal microflora.

9. Pharmaceutical compositions comprising probiotic bacteria strains obtained by the process of claim 1, to treat and/or prevent the alterations of the intestinal microflora.

10. A probiotic food comprising probiotic bacteria strains obtained by the process of claim 1.
